SEQUENCE LISTING (7 sequences)

SEQ ID NO: 1 - protein, 4 aa, Unknown
  endoplasmic retention signal sequence
KDEL

SEQ ID NO: 2 - protein, 5 aa, Pseudomonas sp.
REDLK

SEQ ID NO: 3 - protein, 7 aa, Artificial Sequence
  synthetic variant
ETAKDEL

SEQ ID NO: 4 - DNA, 39 bp, Artificial Sequence
  CD33 PCR primer
ggcagggcggcccagccggccgatccaaatttctggctg

SEQ ID NO: 5 - DNA, 28 bp, Artificial Sequence
  CD33 PCR primer
ctccgcggccgccatgaaccactcctgc

SEQ ID NO: 6 - DNA, 15 bp, Artificial Sequence
  Template PCR primer
cgcgctcgagcctgc

SEQ ID NO: 7 - DNA, 51 bp, Artificial Sequence
  Template PCR primer
ccaaagctcagcaagctttcattacagctcgtccttcggcggtttgccggg

(19) United States
(12) Patent Application Publication
(10) Pub. No.: US 2009/0297521 A1
(43) Pub. Date: Dec. 3, 2009

(54) CD33-SPECIFIC SINGLE-CHAIN IMMUNOTOXIN AND METHODS OF USE

(76) Inventors: Georg H. Fey, Neunkirchen (DE); Michael Schwemmlein, Rengensburg (DE)

Correspondence Address: PERKINS COIE LLP, P.O. BOX 1208, SEATTLE, WA, 98111-1208, US

(21) Appl. No.: 11/997,246

(22) PCT Filed: Jul. 31, 2006
(86) PCT No.: PCT/EP06/07580

§ 371 Date: Jun. 19, 2009

Related U.S. Application Data
(60) Provisional application No. 60/703,692, filed on Jul. 29, 2005.

(51) Int. Cl. A61K 39/395; C07K 16/18; C12N 5/08; A61P 35/00

(52) U.S. Cl. 424/134.1; 530/387.3; 435/366

(57) ABSTRACT

A single-chain immunotoxin composition and method of treatment with the composition is disclosed. Preferably, the immunotoxin is comprised of a CD33-specific single chain Fv antibody fragment and a genetically engineered variant of Pseudomonas Exotoxin A (ETA). A preferred engineered Exotoxin A is referred to ETA′ and may includes a KDEL peptide at its C-terminus, a cellular peptide mediating improved retrograde transport to the endoplasmic reticulum (ER). The immunotoxin compound may be formulated with a carrier and administered into patients where the antibody portion binds to CD33-positive cells and kills those cells to provide an effective treatment for diseases such as human myeloid leukemia.

### FIELD OF THE INVENTION

This invention relates generally to pharmaceutical formulations and more specifically to formulations comprised of an active ingredient which binds to a cell surface antigen on an abnormal cell and causes cell death thereby providing a formulation useful in the treatment of patients with such abnormal cells.

### BACKGROUND

Acute myeloid leukemia is the most common acute leukemia in adults with approximately 12,000 new cases per year in the United States (Jemal et al., CA Cancer J Clin, 54:8-29, 2004). Approximately 70-80% of all patients achieve a complete remission after high-dose chemotherapy, but relapses frequently occur (Loewenberg et al., N Engl J Med, 341:1051-1062, 1999). Due to such relapses the resulting overall 5-year survival is only 22% of all patients. The prognosis for patients older than 55 years is even less favorable (Appelbaum et al., Hematology Am Soc Hematol Educ Program, 62-86, 2001). Further, treatment of AML in the elderly has been problematic, because the toxicity of standard chemotherapy is poorly tolerated in the older age group (Loewenberg et al., N Engl J Med, 341:1051-1062, 1999).

One of the antigens most frequently associated with AML is CD33, a 67 kDa glycoprotein (Freeman et al., Blood, 85:2005-2012, 1995). This antigen mediates anti-proliferative and pro-apoptotic effects on primary AML cells when engaged with antibodies (Mingari et al., Immunol Rev, 181:260-268, 2001). This effect was mediated by its ITIM-motifs contained in the cytoplasmic domain (Paul et al., Blood, 96:483-490, 2000). The antigen CD33 is expressed during myeloid differentiation and is present on leukemic blasts in 90% of patients with AML, but is expressed neither on normal hematopoietic stem cells nor in non-hematopoietic tissues (Dinndorf et al., Blood, 67:1048-1053, 1986). The expression profile of CD33 and its ability to be rapidly internalized (van der Velden et al., Blood, 97:3197-3204, 2001) make CD33 a suitable target antigen for antibody-based AML therapies. This has been shown in several approaches (Caron et al., Cancer, 73:1049-1056, 1994; Wellhausen et al., J Biol Regul Homeost Agents, 16:139-143, 2002; Appelbaum et al., Semin Hematol, 36:2-8, 1999).

In 2000, Gemtuzumab Ozogamicin (GO, MYLOTARG®, CMA-676; Sievers et al., Blood, 93:3678-3684, 1999) was approved for the treatment of patients with AML. GO consists of a humanized anti-CD33 IgG-antibody, which is chemically coupled to the cytotoxic agent calicheamicin (Hamann et al., Bioconjug Chem, 13:47-58, 2002).

Binding of GO to CD33 results in internalization and intracellular release of calicheamicin (van der Velden et al., Blood, 97:3197-3204, 2001). The calicheamicin attaches to the minor groove of DNA and introduces double-strand DNA breaks (Zein et al., Science, 240:1198-1201, 1988). In phase II clinical trials GO produced a 30% overall response rate in relapsed AML patients (Larson et al., Leukemia, 16:1627-1636, 2002; Sievers et al., Expert Opin Biol Ther, 1:893-901, 2001). However, hepatotoxicity, including severe hepatic veno-occlusive disease, pulmonary toxicity, and severe hypersensitivity reactions involving the respiratory and cardiovascular systems arose in association with the use of GO (Bross et al., Clin Cancer Res, 7:1490-1496, 2001). Antigen-independent cytotoxic activity of GO on CD33-negative ALL-derived cell lines has also been observed (Jedema et al., Leukemia, 18:316-325, 2004).

The present invention endeavors, in part, to solve these problems and provide a useful, compound, formulation and method of treatment.

### BRIEF SUMMARY

A fusion protein is disclosed which is comprised of an antibody fragment portion and a modified toxic portion. Preferably, the antibody fragment portion and the modified toxic portion are connected by a stable peptide bond.

In an embodiment, the antibody binds to a cell surface receptor which is generally expressed on cells being targeted. In one embodiment, the antibody fragment portion is a scFv fragment. In another embodiment, the antibody fragment portion is comprised of a heavy chain and light chain of a scFv antibody fragment. In a related embodiment, the antibody fragment portion is stabilized by a disulfide bond connected between the heavy and light chains. In a preferred embodiment, the antibody fragment specifically binds to CD33.

In one embodiment, the modified toxic protein is an engineered variant of Pseudomonas Exotoxin A (ETA). In a preferred embodiment, the variant is the ETA′ variant, which lacks at least one binding domain of the native ETA. In further preferred embodiment, the modified toxic portion of consists of domains II and III of the Pseudomonas toxin.

In a further embodiment, the immunotoxin may comprise a peptide that enhances movement of the fusion protein to the targeted cells' endoplasmic reticulum. In a preferred embodiment, the peptide comprises KDEL (SEQ ID NO:1) at the C-terminus of the immunotoxin.

In an embodiment, the antibody fragment portion is bound to the toxic portion with a stable peptide bond between the antibody portion and the toxin moiety which allows for the toxic component to be released predominantly inside the cell by mechanisms also utilized by the wild type toxin.

Another aspect of the invention relates to a formulation comprising a pharmaceutically acceptable carrier and a fusion protein comprised of an antibody fragment portion and a modified toxic protein portion. The antibody fragment portion is preferably a scFv antibody fragment which binds to a cell surface antigen such as CD33. The modified toxic protein portion is preferably bound to the antibody fragment by a stable peptide bond. This modified toxic protein, in an embodiment, is a genetically engineered variant of Pseudomonas Exotoxin A (ETA). Preferably, the variant is the ETA′ variant consisting of domains II and III of the Pseudomonas Exotoxin A (ETA). In embodiments, the toxin portion may further be modified to include the tetrapeptide KDEL (SEQ ID NO:1) at its C-terminus. In an additional embodiment, the antibody portion binds CD33 with a binding affinity of 1×10⁻⁶ M or greater affinity. In a further embodiment, the fusion protein is present in the formulation in a concentration of about 0.1 mg/ml to about 100 mg/ml.

In yet another aspect, a method of treatment is disclosed comprising diagnosing a patient with a disease associated with cells expressing a CD33 cell surface antigen; and administering to the patient a therapeutically effective amount of a formulation comprising a pharmaceutically acceptable carrier and a fusion protein comprising a scFv antibody portion which specifically binds to CD33 bound to a toxic protein portion. In embodiments, the fusion protein may include a peptide bond between the antibody and toxic portion and/or a C-terminal sequence comprised of KDEL tetra peptide (SEQ ID NO:1). In one embodiment, the disease is acute myeloid leukemia (AML). In another embodiment, the disease is pediatric acute lymphoblastic leukemia (ALL). In an embodiment, the patient who is diagnosed with AML is a patient experiencing a relapse of AML after at least one prior treatment. In a further embodiment, the method includes repeatedly administering the formulation over a period of time and/or monitoring the patient over the same period of time.

In yet another aspect, a method of inducing apoptosis of human cells is disclosed comprising contacting cells with a fusion protein wherein the fusion protein comprises an antibody fragment portion which binds to any of the cells with a binding affinity of 1×10⁻⁶ M or higher affinity, which antibody portion is bound to a modified toxic protein by a stable peptide bond. In an embodiment, the modified toxic protein portion is a variant of a Pseudomonas Exotoxin A (ETA′). In another embodiment, the ETA′ lacks the authentic binding domain of the intact ETA. In a further embodiment, ETA′ is further comprised of a tetrapeptide KDEL (SEQ ID NO:1). In yet another embodiment, the cells are from a cell line selected from the group consisting of U937, HL-60 and THP-1. In a further embodiment, the antibody fragment portion binds to CD33 with a binding affinity of about 1×10⁻⁷ M. In an additional embodiment, the fusion protein is contacted with the cells at a concentration in the range of about 50 ng/ml to about 2,000 ng/ml.

These and other objects, advantages, and features of the invention will become apparent to those persons skilled in the art upon reading the details of the compounds, formulations and methods as more fully described below.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1 is a schematic representation of the recombinant immunotoxin CD33-ETA′ including STREP, an N-terminal STREP tag; 6×His, hexahistidine tag; the V_{L} and V_{H}, variable region light and heavy chains of the CD33-specific scFv; 4(G₄S), flexible linkers consisting of glycine and serine residues; ETA′, truncated ETA fragment consisting of domains II and III of the Pseudomonas toxin; and KDEL, an ER retention motif. Molecular masses of the fragments in kDa were calculated from their amino acid sequences.

FIG. 2A is an image of a Western blot analysis of the recombinant immunotoxin eluted from streptactin beads using an anti-His-antibody; Lanes 1-4 are elution fractions 1-4; and FIG. 2B is an image of a Coomassie stained polyacrylamide gel showing the purity of the purified recombinant immunotoxin (lanes numbered as in FIG. 2A).

FIGS. 3A, 3B, 3C and 3D are graphs of the number of cells versus the fluorescence intensity showing specific binding of recombinant immunotoxins to antigen-positive cells. Cells were stained with purified scFv-ETA′ fusion proteins (black) or a nonrelated scFv-ETA′ fusion protein (white) at the same concentration and analyzed by FACS. FIG. 3A shows results for CD33-positive U937 cells stained with CD33-ETA′. FIG. 3B shows results for CD33-negative CEM cells stained with CD33-ETA′. FIG. 3C shows results for CD19-positive Namalwa cells stained with CD19-ETA′. FIG. 3D shows results for CD19-negative U937 cells stained with CD19-ETA′.

FIG. 4 is a graph showing the results of how CD33-ETA′ induces cell death of CD33-positive U937 cells at low concentrations but not of CD33-negative CEM and Namalwa cells. U937 (♦), CEM (▪) and Namalwa (▴) cells were treated with single doses of the indicated concentrations of CD33-ETA′ for 72 h. Aliquots of cells were evaluated for percentage of cell death by PI staining of nuclei and FACS analysis. Data points are mean values from three independent experiments. Values reaching statistical significance (P<0.05) are indicated by an asterisk. P values are given for differences in the extent of cell death compared with the untreated control.

FIG. 5 is a graph of results showing how cell death by CD33-ETA′ is blocked by the parental CD33-scFv. U937 cells were treated with PBS (◯), single doses of 100 ng/ml CD33-ETA′ (⋄), 100 ng/ml CD33-ETA′+CD33-scFv (Δ), 100 ng/ml CD33-ETA′+isotype control scFv (▴), CD33-scFv (□) or isotype control scFv (♦) at time point 0. At the indicated time points, viable cells were counted by trypan blue exclusion. Triplicate samples were measured for each time point. The values given are representative of three separate experiments.

FIGS. 6A-6E are images wherein cells were stained with Annexin V and PI at the indicated time points. FIGS. 6A-6B shows results where U937 (FIG. 6A) and CD33-negative CEM cells (FIG. 6B) were treated with single doses of 100 ng/ml CD33-ETA′. FIGS. 6C-6E shows results where U937 (FIG. 6C), HL-60 (FIG. 6D) and THP-1 (FIG. 6E) cells were treated with single doses of 100 ng/ml CD33-ETA′ or CD19-ETA′ (U937, THP-1) or with 500 ng/ml CD33-ETA′ or CD19-ETA′ (HL-60), respectively. Numbers in the bottom right quadrant of each plot represent the percentage of cells in early apoptosis (Annexin V-positive and PI-negative). Numbers in the upper right quadrant of each plot represent the percentage of dead cells (Annexin V-positive and PI-positive). The data are representative of three separate experiments.

FIG. 7 is a graph of results wherein CD33-ETA′ induces cell death of primary patient-derived AML cells. MNCs isolated from bone marrow of patient 1, containing ˜50% CD33-positive cells, were left untreated (♦) or were treated with single doses of 500 ng/ml CD33-ETA′ (▴), 500 ng/ml CD33-ETA′+CD33-scFv (▪) or 500 ng/ml CD33-ETA′+isotype control scFv (). The percentage of dead cells was measured by determination of Annexin V-positive cells by FACS analysis after Annexin V and PI staining. Triplicate samples were measured for each time point.

FIGS. 8A and 8B show results of the elimination of primary patient-derived AML cells by CD33-ETA′. FIG. 8A shows the effect of CD33-ETA′ on MNCs isolated from peripheral blood of patient 2, containing ˜50% CD33-positive cells, and FIG. 8B shows the effect of CD33-ETA′ on MNCs from bone marrow of patient 10, containing ˜25% CD33-positive cells. The cells were left untreated (♦) or were treated with single doses of 500 ng/ml CD33-ETA′ (▴) or 500 ng/ml CD19-ETA′ (▪). The percentage of dead cells was measured by determination of Annexin V-positive cells by FACS analysis after Annexin V and PI staining. Triplicate samples were measured for each time point.

FIGS. 9A and 9B show a solid-phase diagnostic device for determining CD33 levels in a human patient, at initial (9A) and final stages (9B) of the assay.

FIG. 10 shows a portion of a gene chip useful for diagnosing genetic predisposition to cancer, constructed in accordance with the present invention.

### DETAILED DESCRIPTION

Before the present compound formulations and methods are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a protein” includes a plurality of such proteins and reference to “the formulation” includes reference to one or more formulations and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### A. DEFINITIONS

“Operably linked” refers to juxtaposition such that the normal function of the components can be performed. Thus, a coding sequence “operably linked” to control sequences refers to a configuration wherein the coding sequence can be expressed under the control of these sequences and wherein the DNA sequences being linked are contiguous and, in the case of a secretory leader, contiguous and in reading phase. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Linking is accomplished by ligation at convenient restriction sites. If such sites do not exist, then synthetic oligonucleotide adaptors or linkers are used in accord with conventional practice.

“Control sequences” refers to DNA sequences necessary for the expression of an operably linked coding sequence in a particular host organism. The control sequences that are suitable for prokaryotes, for example, include a promoter, optionally an operator sequence, a ribosome binding site, and possibly, other as yet poorly understood sequences. Eukaryotic cells are known to utilize promoters, polyadenylation signals, and enhancers.

“Expression system” refers to DNA sequences containing a desired coding sequence and control sequences in operable linkage, so that hosts transformed with these sequences are capable of producing the encoded proteins. To effect transformation, the expression system may be included on a vector; however, the relevant DNA may then also be integrated into the host chromosome.

As used herein, “cell,” “cell line,” and “cell culture” are used interchangeably and all such designations include progeny. Thus, “transformants” or “transformed cells” include the primary subject cell and cultures derived therefrom without regard for the number of transfers. It is also understood that all progeny may not be precisely identical in DNA content, due to deliberate or inadvertent mutations. Mutant progeny that have the same functionality as screened for in the originally transformed cell are included. Where distinct designations are intended, it will be clear from the context.

“Plasmids” are typically circular double-stranded DNA molecules capable of autonomous replication. As used herein, plasmids are designated by a lower case p preceded and/or followed by capital letters and/or numbers. The starting plasmids herein are commercially available, are publicly available on an unrestricted basis, or can be constructed from such available plasmids in accord with published procedures. In addition, other equivalent plasmids are known in the art and will be apparent to the ordinary artisan.

“Digestion” of DNA refers to catalytic cleavage of the DNA with an enzyme that acts only at certain locations in the DNA. Such enzymes are called restriction enzymes and the sites for which each is specific is called a restriction site. The various restriction enzymes used herein are commercially available and their reaction conditions, cofactors, and other requirements as established by the enzyme suppliers are used. Restriction enzymes commonly are designated by abbreviations composed of a capital letter followed by other letters representing the microorganism from which each restriction enzyme originally was obtained and then a number designating the particular enzyme. In general, about 1 mg of plasmid or DNA fragment is used with about 1-2 units of enzyme in about 20 ml of buffer solution. Appropriate buffers and substrate amounts for particular restriction enzymes are specified by the manufacturer. Incubation of about 1 hour at 37° C. is ordinarily used, but may vary in accordance with the supplier's instructions. After incubation, protein is removed by extraction with phenol and chloroform, and the digested nucleic acid is recovered from the aqueous fraction by precipitation with ethanol. Digestion with a restriction enzyme infrequently is followed with bacterial alkaline phosphatase hydrolysis of the terminal 5′ phosphates to prevent the two restriction cleaved ends of a DNA fragment from “circularizing” or forming a closed loop that would impede insertion of another DNA fragment at the restriction site. Unless otherwise stated, digestion of plasmids is not followed by 5′ terminal dephosphorylation. Procedures and reagents for dephosphorylation are conventional (Maniatis et al., 1982, supra).

“Recovery” or “isolation” of a given fragment of DNA from a restriction digest means separation of the digest on polyacrylamide or agarose gel by electrophoresis, identification of the fragment of interest by comparison of its mobility versus that of marker DNA fragments of known molecular weight, removal of the gel section containing the desired fragment, and separation of the gel from DNA. This procedure is known generally (for example, Lawn et al., Nucleic Acids Res., 9:6103-6114, 1981, and Goeddel et al., Nucleic Acids Res., 8:4057, 1980).

“Ligation” refers to the process of forming phosphodiester bonds between two double stranded nucleic acid fragments (Maniatis et al. 1982, supra, p. 146). Unless otherwise provided, ligation may be accomplished using known buffers and conditions with 10 units of T4 DNA ligase (“ligase”) per 0.5 mg of approximately equimolar amounts of the DNA fragments to be ligated.

“Preparation” of DNA from transformants means isolating plasmid DNA from microbial culture. Unless otherwise provided, the alkaline/SDS method of Maniatis et al., supra, p. 90, may be used.

“Oligonucleotides” are short-length, single- or double-stranded polydeoxynucleotides that are chemically synthesized by known methods (such as phosphotdester, phosphite, or phosphoramidite chemistry, using solid phase techniques such as described in EP Pat. Pub. No. 266,032, or via deoxynucleoside H-phosphonate intermediates as described by Froehler et al. (Nucl. Acids Res., 14:5399-5407, 1986). They are then purified on polyacrylamide gels.

“Screening” for cancer, in accordance with the present invention, means testing individuals for a level of CD33 that is indicative of cancer or an elevated risk of cancer.

“Staging” treatment of cancer, in accordance with the present invention, involves determining the stage of cancer in an individual, based on the level of CD33 detected, and tailoring the treatment to that stage. There are four recognized stages of cancer, which are defined by the degree of localization of cancer cells. In addition, cancer may be defined as early stage at which the cancer is responsive to a number of hormonal-based therapies, and a later, more serious androgen-independent stage. It should be noted that AML currently does not fall into these recognized stages. Instead, treatment options are generally based on previous treatment, e.g. untreated, remission, and recurrent or refractory to treatment.

“Reduced expression of CD33” may include, as an indicator of cancer, a reduced level of wildtype CD33 protein or a reduced level of CD33 protein having a specific epitope or domain. That is, either the absence of any CD33 protein or the presence of a defective CD33 protein may be indicative of cancer, e.g., AML.

Abbreviations: AML: acute myeloid leukemia; scFv: single chain variable fragment; ETA: exotoxin A; ETA′; modified ETA.

### B. IMMUNOTOXIN

The present invention discloses and describes a CD33-specific single chain immunotoxin for the treatment of diseases characterized by expression of CD33. Preferably, the recombinant toxin is generated by attachment of an anti-CD33 scFv to a truncated version of Pseudomonas exotoxin A.

It will be appreciated that the construction principle of recombinant scFv-ETA′ molecules with regard to CD33 may be applicable to those described with regard to CD22, CD25, CD7 and CD64 antigens (Kreitman et al., N Engl J Med, 345:241-247, 2001; Kreitman et al., J Clin Oncol, 18:1622-1636, 2000; Peipp et al., Cancer Res, 62:2848-2855, 2002; Tur et al., Cancer Res, 63:8414-8419, 2003).

Several of these scFv-ETA′ toxins exhibit their cytotoxic effects in low ranges such as a concentration range of ng/ml including the CD22-ETA′ and CD25-ETA′ toxins (Kreitman et al., N Engl J Med, 345:241-247, 2001; Kreitman et al., J Clin Oncol, 18:1622-1636, 2000) as well as the CD33-ETA′ toxin described herein and other published constructs (Weis et al., Int J Cancer, 60:137-144, 1995; Tur et al., Cancer Res, 63:8414-8419, 2003).

Further, cytolysis by the CD33-ETA′ toxin is highly antigen-specific. The binding affinity of the fusion protein of the invention for the antigen CD33 is 1×10⁻⁶ M or greater affinity or specifically about 1×10⁻⁷ M. Another aspect of the invention is that CD33-ETA′-induced cell death occurred by apoptosis as demonstrated by Annexin V staining.

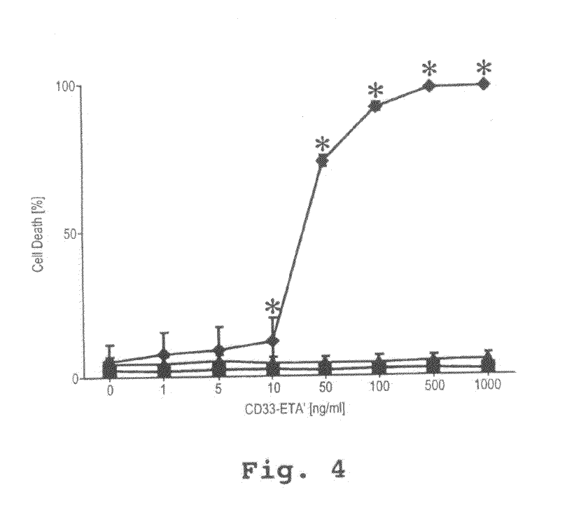

Without being limited as to theory, it is believed that the adverse effects obtained by previous attempts at treatment with immunotoxins may be due to the presence of an Fc-portion in the antibody and the unstable chemical linker between the antibody portion and the toxin moiety resulting in unspecific activity. In an embodiment, the present immunotoxin is a single-chain immunotoxin comprising (1) a single chain Fv antibody fragment and (2) an engineered toxin. In a preferred embodiment, the immunotoxin comprises a CD33-specific single chain Fv antibody fragment and (2) a variant of Pseudomonas Exotoxin A (ETA). The variant toxin preferably carries a cellular peptide mediating transport such as the KDEL peptide (SEQ ID NO:1) at its C-terminus, a cellular peptide mediating improved retrograde transport to the endoplasmic reticulum (ER). As described in Example 1.1, a cDNA coding for Pseudomonas Exotoxin A (ETA) was obtained against an MTA. Anti-CD33 scFv-ETA′ recombinant immunotoxin was constructed, expressed in E. Coli, and purified. The CD33-ETA′ was tested with each of a CD33-positive human monocytic cell line (U937), and CD33-negative CEM cells. As seen in FIGS. 3A-3B, the CD33-ETA′ specifically reacted with the CD33-positive U937 cells, but failed to react with CD33-negative CEM-cells, a cell line derived from a human acute T-cell leukemia (T-ALL), which was reactive with a similarly constructed CD7-specific immunotoxin (Peipp et al., Cancer Res, 62:2848-2855, 2002).

For comparison, the similarly constructed CD19-specific ETA′-immunotoxin (termed CD19-ETA′) reacted with CD19-positive Namalwa cells, a cell line derived from human Burkitt lymphoma (FIG. 3C), but failed to react with the CD19-negative U937 cells (FIG. 3D).

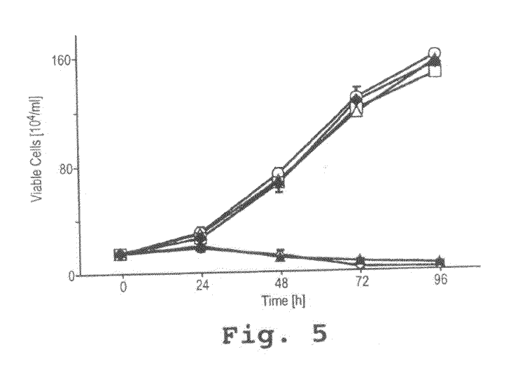

As described in Example 1.2, the CD33-ETA′ immunotoxin mediates specific death of CD33-positive cells, but not CD33-negative cells. As seen in FIG. 4, the mediated specific death of cultured CD33-positive U937 cells, but failed to eliminate CD33-negative CEM and Namalwa cells. As seen in FIG. 4, cell death for the U937 cells (CD33⁺) were near 100% at concentrations of immunotoxin above 100 ng/ml. In contrast, cell death for the CD33-negative CEM and Namalwa cells was less than 10% at all concentrations of the immunotoxin. These results confirm the ability of CD33-ETA′ to effect cell death in a highly antigen-specific manner for a variety of different CD33-positive tumor-derived human cell lines representing different disease entities.

Fifty percent cellular lysis was obtained after 72 h with a single dose of 30-40 ng/ml, corresponding to approximately 0.5 nM. Complete lysis within 72 h was achieved with a single dose of 500 ng/ml (7 nM, P<0.002). Statistically significant lysis (P<0.04) in comparison with untreated controls was obtained with single doses of ≧10 ng/ml. Thus, CD33-ETA acts in a highly antigen-specific manner and is effective for cultured malignant cells in the low nanomolar concentration range.

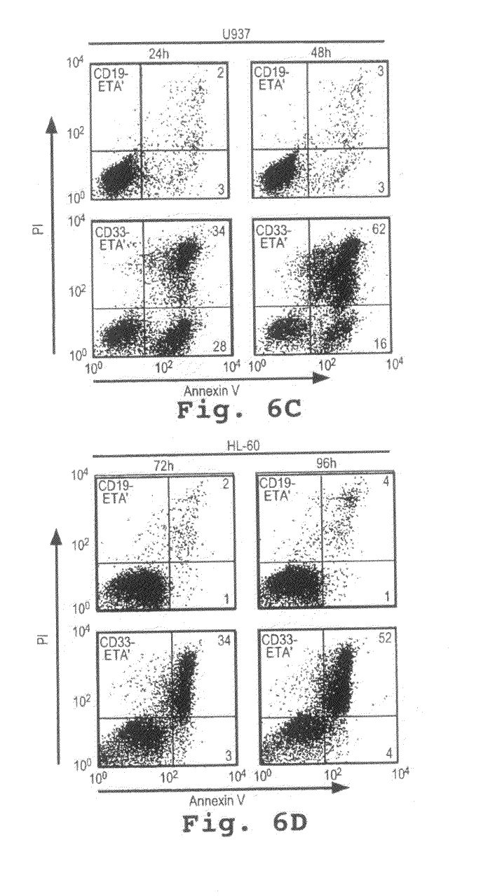

As shown in Example 1.3, formulation of a carrier and purified recombinant fusion protein caused apoptosis of human AML-derived cell lines U937, HL-60 and THP-1. The formulation killed nearly all U937 cells after 72 h with a single-dose of 500 ng/ml, corresponding to ˜7 nM (FIG. 6A). Killing was antigen-specific and mediated by apoptosis. This method of Annexin V and PI staining provides independent evidence for cell death by apoptosis beyond the method of counting cells with SubG1-DNA content presented above. For comparison, no significant death of CD33-negative, CD7-positive CEM cells was mediated by CD33-ETA′ (FIG. 6B), whereas these cells were fully susceptible to lysis by the corresponding CD7-ETA′ toxin (Peipp et al., Cancer Res, 62:2848-2855, 2002).

1. Antibody

In a preferred embodiment, the antibody portion of the immunotoxin is an antibody fragment. More preferably, the antibody fragment is a single chain Fv antibody fragment (scFv). The scFv of an antibody is a fusion of the variable regions of the heavy and light chains of immunoglobulin, linked together with a short (usually serine, glycine) linker. In a preferred embodiment, the scFv is specific to CD33. This section describes production of a CD33 specific single chain Fv antibody fragment useful in the immunotoxin; however, it will be appreciated that other antibody fragments will be applicable to the present invention. CD33 is an antigen expressed by monocytic/myeloid lineage cells including most acute myeloid leukemias. The CD33 gene (also known as GP67) encodes an antigen that is expressed by monocytic/myeloid lineage cells, including most acute myeloid leukemias.

The human CD33 gene (GenBank Accession No. NC_{—}001772) has 7 exons and is localized to the q13.3 region of chromosome 19. The gene encodes a 364 AA, 67 kDa protein (GenBank Accession NP_{—}001763), also designated p67, which is expressed on the surface of normal human myeloid progenitors and leukemic cells from most patients with acute myelogenous leukemia (Peiper, et al., Blood, 72(1):314-21, 1988). The mouse Cd33 gene (NM_{—}021293) is found in chromosome 7; the encoded protein (NP_{—}067268) has 334 AA. The human protein includes a signal peptide (aa 1-17), an extracellular region of 241 residues that includes and IgV domain (aa 18-121) and an IgC2 type domain (aa 156-219), a transmembrane spanning domain (aa 260-282), and a cytoplasmic tail (aa 283-364). When analyzed without reduction, the molecule appears to be a homodimer.

The CD33 antibody used in the present invention can be obtained by any variety of conventional methods to produce a monoclonal, polyclonal, and/or recombinant antibody. Murine and human CD33 antibodies are further available commercially (Becton Dickinson, New Jersey). In one embodiment, the CD33 antibody is a human CD33 antibody. This antibody may be obtained, for example, by expressing the CD33 gene. The purified CD33 protein acts as an immunogen. Alternatively, a partial peptide of CD33 can be used as a sensitization antigen. In particular, for generating antibodies specific against a selected epitope or domain of CD33, a peptide defining that domain or epitope may be used as the immunogen. In another embodiment, the antibody is a mouse monoclonal antibody, prepared according to well-known hybridoma methodology.

Anti-CD33 antibodies may be labeled with a variety of detectable labels, including detectable reporters, such as enzymes for enzyme-linked immunosorbent assays (ELISA), detectable particles, such as gold particles and reporter-carrying liposomes, colorimetric or fluorescent reporters, labels such as quantum dot nanocrystal particles, radiolabels, and labels such as a biotin label by which secondary detectable labels, such as a reporter-labeled streptavidin label can be attached. In some assay formats, an unlabeled anti-CD33 antibody, for example, a mouse IgG antibody, is detected by reaction with a labeled antibody, e.g., a labeled anti-mouse IgG antibody.

For therapeutic uses, human monoclonal antibodies having binding activity to CD33 can be produced by sensitizing in vitro human lymphocytes with CD33, and causing the sensitized lymphocytes to fuse with the human-derived myeloma cells having a permanent division potential. Alternatively, CD33 as an antigen can be administered to a transgenic animal having all the repertories of a human antibody gene to obtain anti-CD33 antibody-producing cells, and then human antibodies for CD33 may be obtained from the immortalized anti-CD33 antibody-producing cells.

In still other methods, human or humanized antibodies specific against CD33 antigen can be prepared by recombinant techniques, such as have been reported (see, for example, U.S. Pat. Nos. 6,090,382 and 6,258,562).

It will be appreciated that the scFv-component may be stabilized by site-directed mutagenesis and introduction of an additional disulfide bond between the V_{H} and V_{L} chains (M. Schwemmlein, unpublished data). However, it will be appreciated that such stabilized scFv may have reduced affinity.

2. Toxin

The toxin portion of the compound is preferably Pseudomonas Exotoxin A (ETA). More preferably, the toxin portion is a truncated version of ETA lacking domain I and containing only domains II and M. (Weis et al., Int J Cancer, 60:137-144, 1995). Domain I is the binding domain for the a2-macroglobulin receptor (CD91) present on most mammalian cells (Kounnas et al., J Biol Chem, 267:12420-12423, 1992).

Domains II and III of ETA are required for intracellular transport and carry the active center of the toxin, respectively, which inhibit protein synthesis by blocking the translation elongation factor EF-2 and causes apoptosis (Lord et al., Cell Microbiol, 1:85-91, 1999). Consequently, the truncated variant of ETA, abbreviated ETA′, which lacks domain I is not toxic as long as it remains in the extracellular space. In addition, ETA′ can be administered with fewer side effects on vascular endothelial cells, because it has an approximately 1000-fold lower affinity to these cells than, e.g., ricin A (Baluna et al., Proc Natl Acad Sci USA, 96:3957-3962, 1999). It will be appreciated that further toxic components may be used for the generation of immunotoxins (see Schnell et al., Leuk Lymphoma, 30:525-537, 1998; Grossbard et al., J Clin Oncol, 11:726-737, 1993).

The toxic portion of the immunotoxin may further comprise a transport peptide such as KDEL (SEQ ID NO:1), which is a C-terminal characteristic ER retention sequence of a variety of luminal ER proteins (Munro et al., Cell, 48:899-907, 1987). Preferably, in order to generate the scFv-ETA′ immunotoxin, the toxin portion includes the C-terminal KDEL motif. Replacing the native REDLK C-terminal sequence (SEQ ID NO:2) of ETA′ by the KDEL sequence (SEQ ID NO:1) has been reported to increase cytotoxicity of ETA′ toxins up to 5-fold (Seetharam et al., J Biol Chem, 266:17376-17381, 1991).

It will be appreciated that in an embodiment, the recombinant toxin portion of the invention can only be absorbed by CD33-positive cells by virtue of its scFv domain and the internalization mechanisms of these cells, triggered by occupation of the CD33 antigen with an antibody or antibody-fragment.

### C. TREATMENT METHODS AND PHARMACEUTICAL PREPARATIONS

The invention also includes methods for treating, e.g., reducing the tumor burden in a human subject with a cancer characterized by expression of CD33. The section below is described in relation to acute myeloid leukemia; however, it will be appreciated that the method may be practiced for other cancers involving expression of CD33.

Coupling of a scFv antibody fragment directed against an antigen capable of internalization to the ETA′ variant results in a potent immunotoxin. There is evidence that similar CD22- and CD25-specific constructs can be used for the treatment of hairy cell leukemias and CD25-positive hematological malignancies (Kreitman et al., N Engl J Med, 345:241-247, 2001; Kreitman et al., J Clin Oncol, 18:1622-1636, 2000).

For therapeutic applications, the fusion protein of the present invention (such as CD33-ETA toxin) is formulated and administered to a mammal, preferably a human, in a pharmaceutically acceptable dosage form, including those that may be administered to a human intravenously as a bolus or by continuous infusion over a period of time, by intramuscular, intraperitoneal, intracerebrospinal, subcutaneous, infra-arterial, intrasynovial, intrathecal, oral, topical, or inhalation routes. The fusion proteins of the present invention are also suitably administered by intratumoral, peritumoral, intralesional or perilesional routes, to exert local as well as systemic effects. Intravenous injection is expected to be particularly useful, for example, in the treatment of acute myeloid leukemia and pediatric lymphoblastic leukemia.

Such dosage forms encompass may further include pharmaceutically acceptable carriers that are inherently nontoxic and nontherapeutic. Examples of such carriers include ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, and polyethylene glycol. Carriers for topical or gel-based forms of CD33-ETA′ toxin protein include polysaccharides such as sodium carboxymethylcellulose or methylcellulose, polyvinylpyrrolidone, polyacrylates, polyoxyethylenepolyoxypropylene-block polymers, polyethylene glycol and wood wax alcohols. For all administrations, conventional depot forms are suitably used. Such forms include, for example, microcapsules, nano-capsules, liposomes, plasters, inhalation forms, nose sprays, sublingual tablets, and sustained release preparations. Sustained release compositions are known and described in U.S. Pat. No. 3,773,919, EP 58,481A, U.S. Pat. No. 3,887,699, EP 158,277A, Canadian Patent No. 1176565, Sidman et al., Biopolymers 22:547, 1983, and Langer et al., Chem. Tech., 12:98, 1982. The CD33-ETA′ toxin protein will usually be formulated in such vehicles at a concentration of about 0.1 mg/ml to 100 mg/ml.

Optionally other ingredients may be added to pharmaceutical formulations of the fusion proteins of the present invention such as antioxidants, including, but not limited to, ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids such as glycine, glutamic acid, aspartic acid, or arginine; monosaccharides, disaccharides, and other carbohydrates including cellulose or its derivatives, glucose, mannose, or dextrins; chelating agents such as EDTA; and sugar alcohols such as mannitol or sorbitol.

It will be appreciated that some dosage forms of the fusion protein (e.g. injectable CD33-ETA′ toxin) to be used for therapeutic administration must be sterile. Sterility is readily accomplished by filtration through sterile filtration membranes (e.g., 0.2 micron membranes). The fusion proteins ordinarily will be stored in lyophilized form or as an aqueous solution if it is highly stable to thermal and oxidative denaturation. The pH of the fusion proteins preparations typically will be about from 6 to 8, although higher or lower pH values may also be appropriate in certain instances.

For the prevention or treatment of disease, the appropriate dosage of fusion protein such as CD33-ETA′ toxin will depend upon the type of disease to be treated, the severity and course of the disease, whether the fusion proteins are administered for preventative or therapeutic purposes, previous therapy, the patient's clinical history and response to the fusion proteins and the discretion of the attending physician. The fusion protein such as CD33-ETA′ toxin is suitable to be administered to the patient at one time or over a series of treatments. For purposes herein, the “therapeutically effective amount” of a fusion proteins is an amount that is effective to either prevent, lessen the worsening of, alleviate, or cure the treated condition, in particular that amount which is sufficient to reduce or inhibit cells that express a CD33 antigen in vivo.

Specifically, the fusion protein of the invention can be formulated and used to treat diseases such as acute myeloid leukemia (AML) and pediatric acute lymphoblastic leukemia (ALL). When treating such diseases the fusion protein of the present invention is formulated such as into an injectable formulation and administered to the patient based on patient size while the administering caregiver will take into consideration other criteria such as the condition, sex, age and other relevant characteristics of the patient. The dosing may be in a range such as from about 1 microgram per kilogram to about 200 micrograms per kilogram or about 3 micrograms per kilogram of patient body weight to about 50 micrograms per kilogram. The administration may be intravenous infusion every other day for a total of about 3 doses. The cycle may be repeated several times while monitoring patient responsiveness. The patient may be monitored in terms of responsiveness by considering a characteristic such as computed tomography, flow cytometry to detect leukemia antigens and histologic antigens of the patient's bone marrow.

The fusion proteins of the present invention are useful in the treatment of various diseases and disorders. The invention includes a method of treatment, comprising diagnosing a patient with a disease associated with cells expressing a CD33 cell surface antigen. A therapeutically effective amount of a formulation comprising an optional pharmaceutically acceptable carrier and a fusion protein comprising a scFv antibody portion which specifically binds to CD33 which antibody portion is bound by a peptide bond to a toxic protein portion is administered to the patient. The toxic protein portion may further comprise the C-terminal sequence comprised of a KDEL peptide (SEQ ID NO:1). In an aspect of the invention the disease is acute myeloid leukemia (AML). In another aspect of the invention the patient diagnosed with (AML) is a patient experiencing a relapse of AML after a prior treatment. In yet another aspect of the invention the disease is pediatric acute lymphoblastic leukemia (ALL). In still another aspect, the fusion proteins are repeatedly administered to the patient over a period of time, and the patient is monitored over that period of time.

As described in Example 1.3, the scFv-toxin was tested on primary Myeloid Leukemia blasts (AML; from either peripheral blood or bone marrow of AML patients at diagnosis or at relapse, who were not currently undergoing chemotherapy). As seen in Table 1, primary cells from 9 out of 10 patients clearly showed increased lysis in cell culture after treatment with anti-CD33 scFv-ETA.

In yet another aspect, a method of inducing apoptosis of human cells includes contacting a cell line chosen from U937, HL-60 and THP-1 with the fusion protein. In this aspect, the fusion protein comprises an antibody fragment portion which binds to any of the cell lines U937, HL-60 and THP-1 with a binding affinity of 1×10⁻⁶ M or higher affinity. As described above, the antibody portion is bound to a modified toxic protein by a stable peptide bond to form the fusion protein. Preferably, the modified toxic protein portion is a variant of a Pseudomonas Exotoxin A (ETA′) and the ETA′ is further comprised of a tetrapeptide KDEL. Preferably, the antibody fragment portion binds to CD33 with a binding affinity of about 1×10⁻⁶ M or greater affinity or 1×10⁻⁷ M. In an embodiment, the ETA′ portion of the fusion protein lacks a binding domain. Preferably, the fusion protein is contacted with the cells at a concentration in the range of about 50 ng/ml to about 2,000 ng/ml.

As described above, MYLOTARG™ is an adduct, chemically linked, between an intact anti CD33 antibody and the potent toxin calicheamycin that is approved for clinical use for patients over 60 years of age, which are no longer eligible for other treatments, including chemotherapy. MYLOTARG™ does cause side effects, but has clinical benefits, given the absence of alternatives for the indications for which it is approved. The toxicities appear to be due to the instability of the chemical link between calicheamycin and the antibody, and binding of the antibody Fc portion to Fc-receptors on cells other than the leukemia cells. The link between the toxin and the antibody is weak and allows for release of the toxin inside the cell, once it is internalized via the antibody. The toxin moiety must then be cleaved from the antibody in order to reach its intracellular target sites in the nucleus, where it causes DNA strand breaks and cell death. This bond is labile and cleaved at pH values below a certain threshold in the lysosomes. This bond between the antibody portion and the toxin portion of MYLOTARG™ appears to be unstable and this might cause antigen-unspecific lysis of cells. Experiments with cultured cells showed that MYLOTARG™ lysed the CD33-negative T-ALL cell line CEM (derived from an acute T-cell leukemia), thus establishing antigen-unspecific actions.

By contrast, the anti-CD33 scFv-Pseudomonas ETA′ fusion of the present invention specifically only eliminated CD33-positive, but not antigen-negative cells. Others have reported that MYLOTARG™ also killed antigen negative B-lymphoid cell lines in vitro. It is pointed out that MYLOTARG™ must to be used within 7 hrs after resuspension in solution from the original lyophilized agent.

As described in Example 1.3, apoptosis was measured by Annexin V and PI staining after treatment of U937, CEM, and Namalwa cells with a CD33-ETA′ immunotoxin.

CD33-specific toxin was shown to not only mediate cell death of CD33-positive cell lines but also of primary human AML cells, although with lower efficiency. The specificity of the agent was established by two different approaches: (a) killing experiments with CD33-positive and CD33-negative cell lines, and (b) pre-incubation with the parental anti-CD33 scFv and a non-relevant control scFv (anti-CD19).

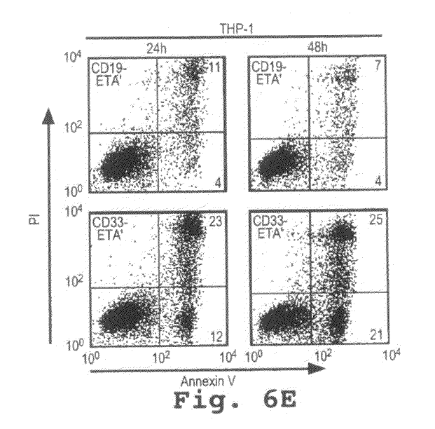

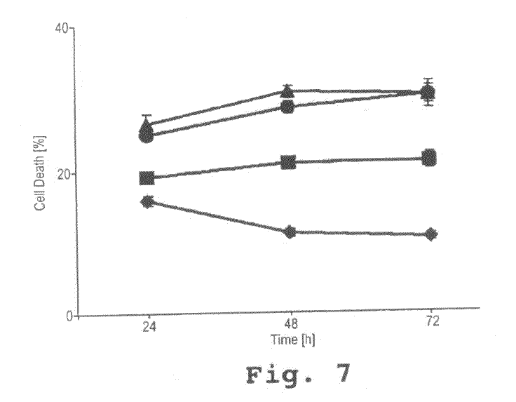

As described in Example 1.4, the immunotoxin also mediated cell death of primary human AML cells, although greater variability of efficacy was observed for a series of samples derived from different patients than for tumor-derived cell lines. In two cases, specific lysis of 20%, and in another case specific lysis of 30% was observed for bone marrow and peripheral blood mononuclear cells (MNCs) obtained from three independent freshly diagnosed AML patients prior to the start of chemotherapy (see FIGS. 7, 8A and 8B).

The data clearly demonstrates that blocking of the CD33 binding sites not only prevents the cells from being lysed by the immunotoxin, but even allows the pretreated cells to proliferate equally fast as the untreated control cells. These experiments show that CD33-ETA′ acts in a highly antigen-specific manner. This property is very pronounced in comparison with GO, because other authors reported killing of CD33-negative cell lines by GO (Jedema et al., Leukemia, 18:316-325, 2004).

In fact, the high degree of antigen-specificity is one of the advantages of the fusion protein, specifically the CD33-ETA′, over the current treatment standard. Another, non-limiting, advantage includes a greater stability, because the toxin component is linked to the scFv-component by a peptide bond instead of a less stable chemical bond used in GO. Moreover, with the CD33-ETA′ fusion protein, the binding domain for the Pseudomonas Exotoxin A receptor on mammalian cells, the a2-macroglobulin receptor (Lord et al., Cell Microbiol, 1:85-91, 1999), is lacking. Therefore, should the toxin component of CD33-ETA′ be released by proteolysis, it would be incapable of binding to the receptor and would therefore not be toxic. By contrast, if the calicheamicin toxin were to be released from the antibody component of GO, it could still exert toxic side-effects on bystander cells.

Another advantage with the present immunotoxin is the stable link between antibody-portion and toxin moiety provided by the peptide bond resulting in reduced non-specific toxicities due to the breakage of this bond in the extracellular space. Also, due to the genetic link between the scFv and the toxin, the population of molecules is very homogeneous at the molecular level, especially as compared to MYLOTARG™, the currently approved treatment for AML. As the immunotoxin is smaller that of the currently available treatment, the immunotoxin is expected to have better biodistribution and pharmakokinetic properties than the larger compound.

Further, due to the absence of the Fc portion in one preferred embodiment, undesirable interactions of the Fc portion with Fc receptors on cells other than the tumor-target cells are prevented. Further, less removal by Fc-receptors on undesirable cells and fewer undesirable side effects should result from the immunotoxin as described.

The fusion protein may be produced in recombinant bacterial systems, therefore, the costs of production (COG; cost of goods) is reduced.

Similar dose limiting toxicities are to be anticipated for CD33-ETA′ as those observed by other authors for scFv-ETA immunotoxins (Kreitman et al., N Engl J Med, 345:241-247, 2001; Kreitman et al., J Clin Oncol, 18:1622-1636, 2000). However, in the experiments performed and described in the examples, side-effects, although existent, were always manageable by the treating physicians.

To demonstrate the characteristics of the immunotoxin, a control protein was constructed and purified which control protein consisted of a CD19-specific single chain Fv antibody fragment fused to the ETA-KDEL toxin as described in Example 1. This control protein failed to induce lysis of the CD19-negative cell lines U937, HL-60 and MP-1. The CD33-ETA toxin also mediated partial lysis of fresh patient-derived AML cells from bone marrow and peripheral blood. In three cases, between 20 and 30% lysis was achieved by treatment of the cells in culture with a single dose of 500 ng/ml. The pronounced antigen-specificity of the protein and formulation of the present invention shows its utility in treating patient's with Myeloid Leukemia. As seen in Table 1, for nine out of ten unrelated freshly diagnosed AML patients with different proportions of CD33-positive cells in their bone marrow and peripheral blood killing with a maximum of 30% was observed.

As seen from the example, CD33-ETA′ eliminated 20-30% of primary AML cells. Under comparable conditions, more than 95% of U937 cells were destroyed. However, it should be noted that U937 cells actively divide in culture with a doubling time of close to 24 h under the experimental conditions as used in the Examples. By contrast, the primary AML cells do not proliferate under the same conditions. Therefore, if action of the agent required successful passage through a whole cell cycle, then only a correspondingly lower extent of cell death would be expected. Resting primary AML cells were reported to be relatively resistant to diphtheria toxin (Jedema et al., Exp Hematol, 32:188-194, 2004), which exhibits its cytotoxic effect by the same mechanisms as Pseudomonas Exotoxin A. It can be presumed that resting AML cells may also be partially resistant to the CD33-ETA′ fusion protein.

Further, completely lysed cells are no longer detectable by FACS analysis as indicated by the decrease in overall cell death measured for the cells of patient 10 after 72 h (FIG. 8B). Finally, the percent cell death described was calculated relative to the total number of patient cells placed into the experiment. However, only a part of these cells were CD33-positive and the exact fraction of malignant blasts has not been evaluated. Isolation of the CD33-positive cell population by, e.g., magnetic cell sorting using magnetic labeled antibodies was not feasible without occupation of CD33 which could result in blocking of the binding site of the immunotoxin and internalization of the target antigen.

The CD33-ETA′ also showed activity in the low nanomolar concentration range. This range is very similar to the range reported by other authors for similarly constructed scFv-ETA′ immunotoxins and GO (Peipp et al., Cancer Res, 62:2848-2855, 2002; Tur et al., Cancer Res, 63:8414-8419, 2003; Amico et al., Blood, 101:4589-4597, 2003).

In one general immunotherapy approach, a patient diagnosed with AML is treated by administration of the anti-CD33 immunotoxin. Preferably the fusion antibody is a human or humanized antibody, prepared as described above, and is administered by IV or subcutaneous injection in a suitable physiological carrier. During treatment, the patient is monitored for change in status of the cancer, typically by a combination of a tumor-visualization procedure and levels of CD33 antigens. The treatment may also be carried out in combination with other cancer treatments, including drug or radioisotope therapy, and may be continued until a desired diminution in tumor size is observed.

### D. DIAGNOSTIC METHODS AND REAGENTS

In one aspect, the invention includes a method of screening for cells expressing CD33 in a human subject. In another aspect, the invention includes a method of staging treatment of cancer expressing CD33 in a subject. This is done, in accordance with the invention, by reacting a body-fluid sample from the subject with an antibody specific against a selected domain or epitope of CD33, and determining from the presence and/or amount of immunoassay product, whether the subject has an increased level of CD33 protein, when compared with a normal range of CD33 in human samples. Increased levels are an indicator of cancer.

The assay may be carried out by any of a variety of assay methods used for detecting body-fluid antigens, including ELISA techniques, homogeneous assays, for example, involving fluorescence quenching, and a variety of solid-phase sandwich assays in which the CD33 antigen is captured by an anti-CD33 antibody carried on a solid support, and the immobilized antigen-antibody complex is labeled with a second anti-CD33 antibody, e.g., a second antibody carrying a colorimetric or gold-particle reporter.

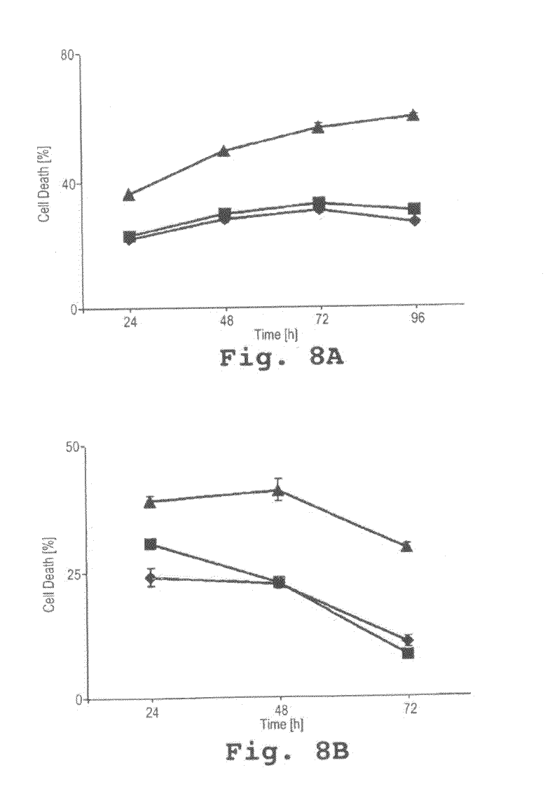

FIGS. 9A and 9B illustrate a solid-phase assay strip constructed in accordance with an embodiment of the invention, suitable for carrying out a sandwich immunoassay of the type just mentioned, and shown in initial and final assay states, respectively. The strip, indicated generally at 10, includes a porous support or pad 12 having a sample-application zone 14 in an upstream region of the support and a sample-detection zone 16 in a downstream region. The sample-application zone includes a detectable anti-CD33 antibody reagent, e.g., anti-CD33 antibodies labeled with gold particles, and carried in the zone in an unbound, i.e., non-immobilized form. This reagent is indicated by solid circles, such as at 18. Anti-CD33 antibodies, which may be the same or different from those in the labeled antibody reagent, are immobilized to the solid support within the detection zone, and are indicated by the “Y” shapes, such as at 20.

Also shown is a reference zone 22 which is located adjacent the detection zone and has one or more colored or shaded regions corresponding to different assay levels of CD33 in a body-fluid sample. In the embodiment shown, zone 22 includes three regions 22 a, 22b, and 22c, corresponding to an assayed level of CD33 (a) below that associated with cancer, (b) corresponding to a lower threshold level associated with cancer, and (c) a level that is substantially higher, e.g., 2-3 times, higher than the threshold layer in region 22b, respectively. These three regions provide a known standard indicator against which the level of detectable reaction produced can be assessed as a level associated with cancer. Together, the assay strip and reference zone constitute an assay device for use in screening for cancer expressing CD33 in a human subject, or for staging treatment of cancer in a human subject.

In operation, a known volume of a body-fluid sample to be tested is added to the sample-application zone of the strip, where it diffuses into the zone, allowing the antibody reagent to react with CD33 antigen in the sample to form an antigen-antibody complex. This complex and unbound antibody reagent then migrate downstream by capillarity toward the detection zone, where the antigen-antibody complex is captured by the immobilize antibody and the unbound reagent is carried to the end of the support, as indicated at 24. As can be appreciated, the higher the concentration of antigen in the body fluid, the higher the density of captured reagent in the detection zone and the greater the color or intensity in this zone. This color or intensity produced in the detection zone is compared with the standards in the reference zone to determine a qualitative level of CD33 associated with the presence or absence of cancer. If an increased level of CD33 is observed in the assay, the subject can be classified in a higher-probability category for the presence of cancer and the subject may be recommended for additional testing and/or more frequent testing.

In another embodiment, the assay device includes an assay strip like that described above, but where the known-reference indicator is provided by a strip-reader instrument reader having (i) a reader slot for receiving the assay strip, (ii) a light source and an optical detection, e.g., a spectrophotometric detector, for detecting an assay-related optical condition at the detection zone of the assay strip, (iii) an electronics or processor unit which records and processes a signal from the optical detector, and converts the signal to an assayed level of CD33, and (iv) a user display screen or window. The instrument may report the actual CD33 body-fluid sample detected, allowing the operator to compare the displayed value with known standard indicator levels provided with the assay strip or instrument, to assess whether the subject has an increased level associated with cancer, or to assess the possible stage of the cancer, for purposes of treatment design. Alternatively, the instrument itself may contain stored known-standard indicator levels which can be compared internally with an assayed level to generate an output that indicates whether an increased CD33 level associated with cancer has been detected, or to indicate the stage of the cancer.

### E. IDENTIFYING GENETIC MUTATION ASSOCIATED WITH CANCER

In another aspect, the invention provides a method for identifying mutations associated with increased risk of cancer, such as AML, in a human subject. The section below is described in relation to AML; however, it will be appreciated that the method may be practiced for other cancers involving expression of CD33. In practicing the method, genomic DNA is extracted from human patients having AML, preferably including patients from men or women representing different racial and age groups. The DNA sequences that are examined, in particular; are (i) one or more of exons 1 to 7 of the CD33 gene on region chromosome q13.3 of chromosome 19, including adjacent splice site acceptor and donor sequences of the exons, (ii) a 5′ UTR region within 10 kB or less of exon 1 of the gene, and (iii) a 3′ UTR region within 10 kB or less of exon 7.

Mutations at one or more sites along the region are identified by comparing each of the sequences with sequences from the same region derived from normal (wildtype) tissue. Preferably sequences from a number of wildtype individuals are determined to ensure a true wildtype sequence. For each extracted DNA, the patient and wildtype sequences are compared to identify mutations in the patient sequences, and thus mutations that are likely associated with increased risk of cancer.

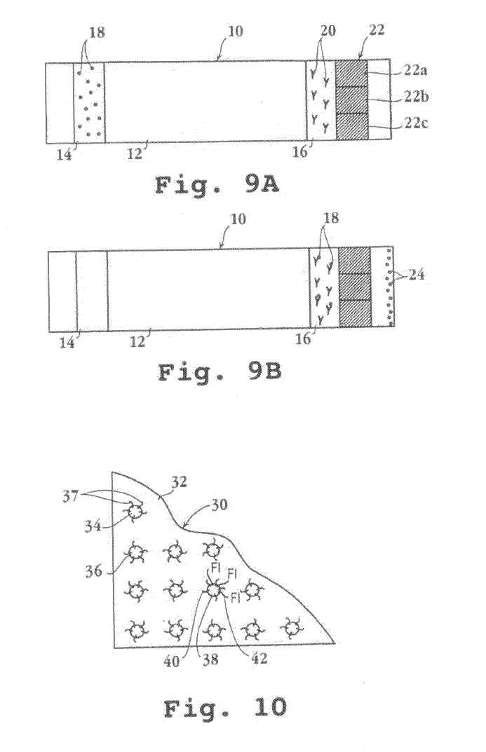

Once a large number of these mutations are identified, e.g., at least 50-200 or more, they may be used in constructing a genetic screening device, e.g., a gene chip, useful for screening individuals for genetic predisposition to cancer. In one embodiment, the device includes a gene chip, such as shown at 30 in FIG. 10, having an array of regions, such as regions 34, 36, each containing bound known-sequence fragments, such as fragment 37 in region 34. The fragments or probes are preferably 25-70 bases in lengths, and each includes one of the above-identified mutations upstream of the CD33 gene that is associated with cancer. Gene-chip construction and detection of mutant sequences with such chips are well known.

In a typical genetic-screening procedure, patient cells are obtained, genomic DNA is extracted, and sequence regions of interest are amplified by standard PCR, employing fluoresceinated probes. The amplified material is then reacted with the chip-array sequences, under suitable hybridization conditions, and the array surface is washed to remove unbound material, and then scanned with a suitable chip reader to identify any mutated sequences associated with cancer. The figure shows binding of a labeled genomic DNA fragment, indicated at 42, to an array region 38 having bound probe molecules 40. Detection of a fluorescent signal in this array region is diagnostic of a known genetic mutation in the critical upstream CD33 region may be diagnostic of a genetic predisposition to AML.

In an alternative embodiment, the mutations identified as above are used to construct a set of molecular inversion probes (MIPs) capable of identifying the presence of genomic mutations. The construction and use of MIPs for identifying genetic mutations have been described (see, for example, Wang, et al., Nucleic Acids Research, (England) 2005, Vol. 33, p. 21).

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Material and Methods

### Bacterial Strains and Plasmids

Escherichia coli XLi-Blue (Stratagene, Amsterdam, the Netherlands) was used for the amplification of plasmids and cloning, and E. coli TG1 (from Dr. G. Winter, MRC, Cambridge, United Kingdom) for screening of antibody libraries. Libraries were generated in the phagemid vector pAK100, and pAK400 was used for the expression of soluble scFvs (Krebber et al., J Immunol Methods, 201:35-55, 1997). E. coli BL21 (DE3; Novagen, Inc., Madison, Wis.) served for the expression of scFv-ETA′ fusion proteins. For the generation of 293T cells expressing human CD33 on the surface, plasmid πH3M (from Dr. Bryan Seed, Massachusetts General Hospital, Boston, Mass.; Simmons et al., J Immunol, 141:2797-2800, 1988) harboring human CD33 cDNA was digested with NotI and HindIII, and the insert was ligated into the vector pcDNA3.1(+) (Invitrogen, Groningen, The Netherlands), resulting in construct pcDNA3.1-hCD33. To express soluble CD33 as a fusion protein with an Fc-portion from a human IgG1 immunoglobulin, a cDNA fragment lacking the transmembrane and intracellular domains of CD33 was amplified by PCR using primers 5′-GGC AGG GCG GCC CAG CCG GCC GAT CCA AAT TTC TGG CTG-3′ (SEQ ID NO:4) and 5′-CTC CGC GGC CGC CAT GM CCA CTC CTG C-3′ (SEQ ID NO:5). The amplified DNA fragment was then ligated into the vector pSecTag-C-Fc containing the coding sequences for the Fc portion of a human IgGI heavy chain as described (Peipp et al., J Immunol Methods, 251:161-176, 2001) resulting in plasmid pSecTag-C-hCD33-Fc.

### Patient Samples and Cell Lines

Heparinized peripheral blood samples and bone marrow samples from AML patients were obtained after receiving informed consent and with the approval of the Ethics Committees of the University of Erlangen-Nuremberg. MNCs were isolated using Percoll separating solution (Biochrom, Berlin, Germany) and were cultured in RPMI 1640-Glutamax-I medium (Invitrogen, Karlsruhe, Germany) containing 20% FCS with or without 50 ng/ml IL-3 and 10 ng/ml GM-CSF (Sigma, Deisenhofen, Germany). Leukemia-derived cell lines U937, HL-60, THP-1, CEM and Namalwa (DSMZ; German Collection of Microorganisms and Cell Lines, Braunschweig, Germany; (Drexler, H. G. The leukemia-lymphoma cell line facts book, San Diego: Academic Press, 2001) were cultured in RPMI 1640-Glutamax-I containing 10% FCS and penicillin and streptomycin (Invitrogen) at 100 units/ml and 100 μg/ml, respectively. Human 293T cells (ATCC) were maintained in DMEM-Glutamax-I medium (Life Technologies, Karlsruhe, Germany) containing 10% FCS, and 100 units/ml penicillin and 100 μg/ml streptomycin.

### Measurement of Cytotoxic Effects of Immunotoxins

For dose response experiments, cells were seeded at 1.5×10⁵/ml in 24-well plates, and immunotoxin was added at varying concentrations. Cell death was measured by staining nuclei with a hypotonic solution of PI as described (Dorrie et al., Cancer Res, 61:4731-4739, 2001; Nicoletti et al., J Immunol Methods, 139:271-279, 1991). The extent of cell death was determined by measuring the fraction of nuclei with subdiploid DNA content. Fifteen thousand events were collected for each sample and analyzed for subdiploid nuclear DNA content. To determine whether cell death was attributable to apoptosis, cells were seeded at 2.5×10⁵/ml and treated with the immunotoxins. Whole cells were stained with FITC-conjugated Annexin V (Pharmingen, Heidelberg, Germany; Vermes et al., J Immunol Methods, 184:39-51, 1995) and PI in PBS according to the manufacturer's protocol. For blocking experiments, cells were seeded at 1.5×10⁵/ml in 24-well plates, and a 100-fold molar excess of the parental scFv antibody or a nonrelated scFv antibody was added to the culture 1 h before adding the immunotoxin. Viable cell counts were determined by trypan blue staining.

### SDS-PAGE and Western Blot Analysis

SDS-PAGE was performed by standard procedures (Laemmli, Nature, 227:680-685, 1970). Gels were stained with Coomassie brilliant blue R250 (Sigma). Western blots were performed with secondary antibodies coupled to horseradish peroxidase (Dianova, Hamburg, Germany; Harlow and Lane, Using Antibodies: A Laboratory Manual. In. Cold Spring Harbor, N.Y.: Cold Spring Harbor Laboratory Press, 1998). Enhanced chemiluminescence reagents (Amersham Pharmacia, Freiburg, Germany) were used for detection. ScFvs and scFv-ETA′ fusion proteins were detected with a penta-His antibody (Qiagen, Inc., Hilden Germany).

### Statistical Analysis

All statistical analyses were performed with Microsoft EXCEL software. P values were obtained using two-tailed paired t tests with a confidence interval of 95% for evaluation of the statistical significance compared to the untreated control.

### Example 1

### Preparation of CD33 scFV-ETA′ Immunotoxin

1. Generation and Specific Binding of the Recombinant Immunotoxin.

A novel CD33-specific single chain Fv (scFv) antibody fragment was generated by immunization of Balb/c mice with a purified recombinant chimeric protein derived from human CD33. To generate the immunogen, the extracellular domain of CD33 was fused to the Fc-portion of a human IgGI antibody to assure solubility and native conformation of the chimeric protein. A phage display library was generated from spleen RNA of the immunized mice and six novel CD33-reactive phages were isolated. The cDNA insert from the most strongly reactive phage isolate was subcloned and fused to the coding sequence for truncated Pseudomonas Exotoxin A lacking the receptor-binding domain. The coding sequence for the C-terminal pentapeptide REDLK (SEQ ID NO:2), a peptide directing the retrograde transport of the authentic toxin, was replaced by the coding sequence for the KDEL-tetrapeptide, a peptide assuring proper retrograde transport of cellular proteins. This replacement was performed following published examples (Brinkmann et al., Proc Natl Acad Sci USA, 88:8616-8620, 1991) to optimize intracellular transport to the ER. The variable light and heavy chain domains (V_{L} and V_{H}) were connected by a sequence coding for the 20 amino acid synthetic linker (G₄S)₄.

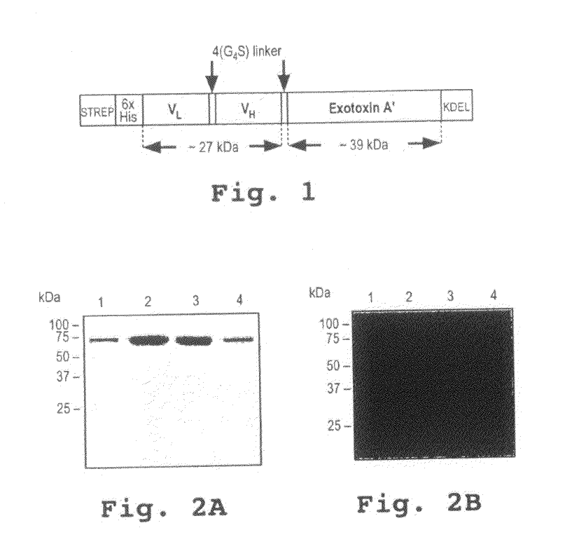

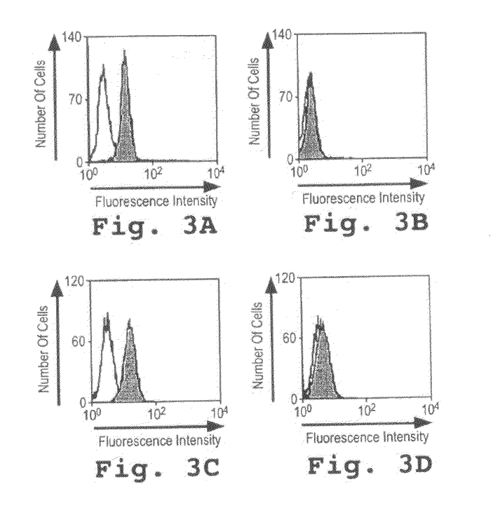

Sequences coding for a STREP-tag and a hexahistidine-tag were added at the N-terminus for detection and purification and a schematic representation of the resulting purified fusion protein is shown in FIG. 1. The resulting polypeptide was expressed in E. coli and purified from periplasmic extracts by affinity chromatography using a streptactin matrix. A single affinity purification cycle resulted in highly enriched protein reactive with an antibody specific for the hexahistidine-tag. Images of a Western blot and a Coomassie stained polyacrylamide gel are shown in FIGS. 2A and 2B, respectively.

The yield was approximately 15-20 μg of purified protein per liter of E. coli culture. For comparison, a CD19-specific immunotoxin containing the ETAKDEL variant (SEQ ID NO:3) was similarly constructed starting from the published murine anti human CD19 hybridoma 4G7 (Meeker et al., Hybridoma, 3:305-320, 1984; Lang et al., Blood, 103:3982-3985, 2004). After expression in E. coli and purification from the periplasm, similar yields were obtained. The CD33-immunotoxin (termed CD33-ETA′) was tested for specific binding of the immunotoxin with CD33-positive human monocytic cell line U937 and CD33-negative CEM cells derived from a human acute T-cell leukemia (T-ALL) with the results shown in FIGS. 3A and 3B, respectively.

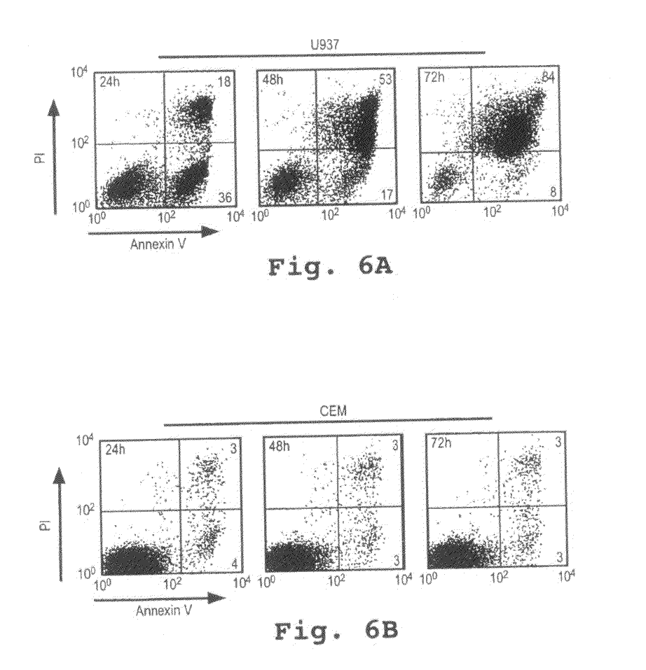

For comparison, a CD19-specific ETA′-immunotoxin (termed CD19-ETA′) was constructed and reacted with CD19-positive Namalwa cells, a cell line derived from human Burkitt lymphoma, and CD19-negative U937 cells with the results shown in FIGS. 3C and 3D. Specificity of binding of CD33-ETA′ was further proven by competition binding studies with large molar excesses of the corresponding parental scFv with the results shown graphically in FIG. 5.

a. Expression and Purification of Soluble CD33-Fc-Fusion Protein

Twenty μg of the expression vector pSecTag-C-hCD33-Fc were transiently transfected in 293T cells using the calcium phosphate procedure including 5 mmol/l chloroquine (Sambrook et al., Molecular Cloning: A Laboratory Manual, Ed. 3. Cold Spring Harbor, N.Y.: Cold Spring Harbor Laboratory Press, 2001). After 12 h, the transfection medium was replaced by fresh culture medium. Supernatants were collected every day for 5 d and combined. Purification was achieved by affinity chromatography with Protein-A agarose beads (Sigma).

b. Generation of CD33 Transfected Cells

To generate 293T cells expressing hCD33 on the surface, 293T cells were transiently transfected with 20 μg of the vector pcDNA3.1-hCD33 as described above. Surface expression of CD33 was verified after 24 h by FACS analysis.

c. Immunization of Mice

BALB/c mice (Charles River, Sulzfeld, Germany) were maintained according to the European guidelines for the protection of laboratory animals. Approximately 60 μg of purified CD33-Fc fusion protein were combined with TiterMax Gold™ adjuvant (Sigma) and injected intraperitoneally on day 0. On days 26 and 41, mice were boosted with 30 μg of protein subcutaneously. A final intraperetoneal injection of 40 μg of protein followed on day 74. Three days later, the mice were sacrificed and spleens were recovered under sterile conditions. The serum was tested at different timepoints for the presence of anti-hCD33 antibodies by FACS analysis using 293T cells that were transiently transfected with hCD33 in the expression plasmid pcDNA3.1-hCD33.

d. Preparation of scFv Phage Display Libraries

Total RNA was prepared with Trizol reagent (Invitrogen) from the spleens of immunized mice according to manufacturers instructions. First-strand cDNA was prepared from 10-15 μg of total RNA (Krebber et al., J Immunol Methods, 201:35-55, 1997). PCR amplification of immunoglobulin variable region cDNAs and cloning into the phagemid vector pAK100 was performed as described (Krebber et al., J Immunol Methods, 201:35-55, 1997; Peipp et al., J Immunol Methods, 251:161-176, 2001). Propagation of combinatorial scFv libraries and filamentous phages was performed by following published procedures (Peipp et al., J Immunol Methods, 251:161-176, 2001).

e. Panning of Phage Display Libraries with Intact Cells

Panning of phage display libraries with intact cells was carried out as described (Peipp, et al, J Immunol Methods, 251:161-176, 2001) using CD33-positive 293T transfectants. Bound phages were eluted with 50 mM HCl. After six rounds of panning, individual phages were purified, and the inserts were sequenced (Sambrook et al., 2001, supra) using an Applied Biosystems automated DNA sequencer (ABI Prism 310 Genetic Analyzer; Perkin-Elmer, Ueberlingen, Germany).

f. Bacterial Expression and Purification of Soluble scFv Antibodies

For the soluble expression of antibody fragments, cDNAs coding for CD33-specific scFvs were subcloned into the expression vector pAK400, and the plasmids were propagated in E. coli HB2151 (from Dr. G. Winter; MRC, Cambridge, United Kingdom). Expression and purification of CD33-specific scFv antibodies was performed as described (Peipp et al., J Immunol Methods, 251:161-176, 2001).

g. Construction and Expression of scFv-ETA′ Fusion Proteins

Sequences coding for the CD33- and CD19-specific scFvs were excised from the pAK400-anti CD33 and pAK400-anti CD19 (Meeker et al., Hybridoma, 3:305-320, 1984) expression constructs harboring the corresponding scFv fragments and were cloned as Sfil-cassettes into the vector pASK-20aa-linker (M. Peipp, unpublished data), a vector containing coding sequences for a N-terminal STREP- and hexa-histidine tag and the 20 amino acid linker (G₄S)₄. The plasmids were digested with NotI and Cel II and the coding sequence for a truncated ETA variant (Peipp et al., Cancer Res, 62:2848-2855, 2002) was ligated into the vectors resulting in the plasmids pASK-STREP-His-CD33-ETA′-REDLK and pASK-STREP-His-CD19-ETA′-REDLK. The sequences coding for both immunotoxins were then cloned into the expression vector pet27b(+) (Novagen, Inc.). For the exchange of the C-terminal REDLK retention motif (SEQ ID NO:3) against the KDEL motif (SEQ ID NO:1), the vector pet27b(+)-STREP-His-CD33-ETA′-REDLK was used as template for PCR reaction using primers 5′-CG CGC TCG AGC CTG C-3′ (SEQ ID NO:6) and 5′-CCA MG CTC AGC AAG CTT TCA TTA CAG CTC GTC CTT CGG CGG TTT GCC GGG-3′ (SEQ ID NO:7). The resulting DNA fragments were digested with Xhol and Cel II and ligated into pet27b(+)-STREP-His-CD33-ETA-REDLK and pet27b(+)-STREP-His-CD19-ETA-REDLK digested with the same restriction enzymes, thereby creating the expression vectors pet27b(+)-STREP-His-CD33-ETA-KDEL and pet27b(+)-STREP-His-CD 19-ETA-KDEL.

The scFv-ETA′ fusion proteins were expressed under osmotic stress conditions as described (Barth et al., Blood, 95:3909-3914, 2000). Induced cultures were harvested 16-20 h after induction. The bacterial pellet from 1 liter culture was resuspended in 200 ml of periplasmatic extraction buffer [100 mM Tris, pH 8.0, 0.5 M sucrose, 1 mM EDTA] for 3 h at 4° C. The scFv-ETA′ fusion proteins were enriched by affinity chromatography using streptactin agarose beads (IBA GmbH, Goettingen, Germany; Skerra et al., Methods Enzymol, 326:271-304, 2000) according to manufacturer's instructions.

h. Flow Cytometric Analysis

The binding of scFvs to cells was analyzed using a FACSCalibur FACS instrument and CellQuest software (Becton Dickinson, Mountain View, Calif.). Cells were stained with scFv antibodies as described (Peipp et al., J Immunol Methods, 251:161-176, 2001). A nonrelated scFv served as a control for background staining. Ten thousand events were collected for each sample, and analyses of whole cells were performed using appropriate scatter gates to exclude cellular debris and aggregates. To monitor binding of scFv-ETA′ fusion proteins, 5×10⁵ cells were incubated for 30 min on ice with 20 μl of the immunotoxin at a concentration of 5 μg/ml. A nonrelated immunotoxin served as a control for background staining. The cells were washed with PBA buffer [containing PBS, 0.1% BSA, and 7 mM Na-azide] and then incubated with 50 μl of a polyclonal rabbit anti-Pseudomonas ETA serum (Sigma) diluted 1:250 in PBA buffer. Cells were washed and incubated with fluorescein-isothiocyanate (FITC)-conjugated pig anti-rabbit-IgG (DAKO Diagnostica GmbH, Hamburg, Germany) for 30 min. After a final wash, cells were analyzed by FACS. For monitoring surface expression of hCD33 on transfected 293T cells, 5×105 cells were incubated for 30 min on ice with 20 μl of anti-hCD33 antibody (Clone WM-54; DAKO Diagnostica GmbH) at a concentration of 1 μg/ml. Mouse IgGI served as an isotype control. Cells were washed in PBA and 20 μl of PE conjugated goat-anti-mouse-IgGI antibody were added. After a final wash, cells were analyzed as described above.

2. Antigen-Specific Cytotoxic Activity of CD33-ETA

The specific death of CD33-ETA′ mediated specific death of cultured CD33-positive U937 cells and CD33-negative CEM and Namalwa cells was measured by nuclear DNA content at 0, 24, 48, 72, and 96 h of treatment, using propidium iodide (PI) staining and flow cytometry with the results being graphed in FIG. 4.

To further verify the antigen-specificity of the immunotoxin, the agent was added to U937 cells and viable cells were counted by using trypan blue exclusion. Cell death was blocked by pretreatment with excess concentrations of the parental CD33-specific scFv antibody. Pre-incubation with a CD19-specific scFv at similar molar excess failed to prevent cellular lysis. In addition, the CD33- and CD19-specific scFvs alone lacking a toxin component did not produce detectable loss of viability. The results are depicted in FIG. 5.

3. Apoptosis of CD33-ETA′ Immunotoxin

To investigate whether death induced by the agent occurred via apoptosis or other cellular routes to elimination, apoptosis was specifically measured by Annexin V and PI staining. Indeed, the passage of cells from an Annexin V⁺PI⁻ (Annexin V positive and PI negative) early apoptotic stage to an Annexin V⁺, PI⁺ late apoptotic state was clearly detectable. As seen in FIGS. 6A-6E, CD33-ETA′ but not CD19-ETA′ induces apoptosis in CD33-positive U937, HL-60 and THP-1 cells. After 24 h of treatment of U937 cells with CD33-ETA′ 36% of early apoptotic and 18% of dead cells were counted, whereas after 48 h and 72 h the corresponding numbers were 17%, 53% and 8%, 84%, respectively (FIG. 6A).

Two additional CD33-positive malignant human cell lines, HL-60 and THP-1, were also killed by CD33-ETA′ via apoptosis, whereas they were refractory to lysis by CD19-ETA′ (FIGS. 6D-6E). However, to establish functionality of the CD19-directed toxin, ALL-derived CD19-positive cell lines Nalm-6 and REH were lysed by CD19-ETA′ in parallel experiments. These results confirm the ability of CD33-ETA′ to kill target cells by apoptosis in a highly antigen-specific manner for a variety of different CD33-positive tumor-derived human cell lines representing different disease entities.

4. Induction of Cell Death of Primary Human AML Cells

To investigate the effect of the agent on primary human cells, MNCs were isolated from the bone marrow of three patients diagnosed with AML. The immunotoxin was then administered to the patient-derived AML cells. Fresh cells were provided by the University Hospital Munich (Klinikum der Innenstadt der LMU; Prof. Bertold Emmerich; Dr. Fuat Oduncu); the University Hospital Erlangen (Medizin Klinik III; Prof. J. Kalden; Dr. B. Stockmeyer) and the Klinikum Nord, City of Nuremberg (Prof. Wilhelm).

In the first case (patient 1), the bone marrow of a patient diagnosed with AML stage FAB M4 contained approximately 50% of CD33-positive cells. Treatment of this mixed population with a single dose of our agent of 500 ng/ml added at time point 0 led to approximately 20% lysis above the background level of spontaneous lysis after 48 h (FIG. 7). Death was prevented by pretreatment of the cells with a 100-fold molar excess of the parental CD33-scFv, but not after pretreatment with an equivalent excess of the non-relevant CD 19-scFv.

Similarly, the peripheral blood of another patient (patient 2) diagnosed with AML stage FAB MI contained approximately 50% of CD33-positive cells. After 96 h in culture, these cells showed 30% killing over the untreated background by a single dose of the agent of 500 ng/ml (FIG. 8A). Bone marrow cells of a third patient (patient 10) diagnosed with AML stage FAB M4 containing approximately 25% of CD33-positive cells showed 19% lysis above the background level of spontaneous lysis after 48 h of treatment with CD33-ETA′ (FIG. 8B). In comparison, the CD19-ETA′ toxin was ineffective against the cells from patients 2 and 3 (FIGS. 8A and 8B). Cells taken from peripheral blood or bone marrow of six other AML patients containing different percentages of CD33-positive cells showed ≦10% lysis above the background level after treatment with the immunotoxin. Treatment of the cells of patient 9 with a second 500 ng/ml dose of the immunotoxin after 48 h and incubation of the cells of patients 7-10 with 50 ng/ml Interleukin-3 (IL-3) and/or 10 ng/ml Granulocyte-Macrophage Colony-Stimulating Factor (GM-CSF) did not increase cellular lysis by CD33-ETA′ (see Table 1 below). Bone marrow cells from one patient (patient 5) did not respond to CD33-ETA′.

TABLE 1
Cytotoxic activity of CD33-ETA′ on
MNCs of different AML patients FAB

| | FAB | | | |
|---|---|---|---|---|
| Patient # | Classification | Source | % CD33+ | % cell death |
| 1 | M4 | BM | ~50 | 20 |
| 2 | M1 | PB | ~50 | 30 |
| 3 | M1 | PB | ~65 | 9 |
| 4 | M4/M5 | PB | ~70 | 9 |
| 5 | M6 | BM | ~80 | 0 |
| 6 | M4 | BM | ~80 | 6 |
| 7 | M0 | BM | ~25 | 5 * |
| 8 | M1 | PB | ~85 | 5 * |
| 9 | M4/M5 | BM | ~85 | 10 * ^{†} ^{‡} |
| 10 | M4 | BM | ~25 | 19 * ^{†} |

* cells were treated with GM-CSF (10 ng/ml);
^{†} cells were treated with IL-3 (50 ng/ml);
^{‡} cells were treated with a second dose of CD33-ETA′ after 48 h;
BM, bone marrow;
PB, peripheral blood

It will be appreciated that embodiments described with respect to one aspect may be applicable to each aspect of the compositions and methods described. It will further be appreciated that embodiments may be used in combination or separately. It will also be realized that sub-combinations of the embodiments may be used with the different aspects. Although the embodiments have been described with many optional features, these features are not required unless specifically stated.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. An fusion protein, comprising:
a single chain scFv antibody fragment portion which specifically binds to cell surface antigen CD33 and is capable of being internalized into a CD33 expressing cell; and a toxic protein portion.

2. The fusion protein according to claim 1, wherein the scFv antibody fragment portion is linked to the toxic protein portion.

3. The fusion protein according to claim 1, wherein the fusion protein further comprises a transport peptide which facilitates movement into a cell's endoplasmic reticulum.

4. The fusion protein according to claim 3 wherein the transport peptide comprises the sequence of SEQ ID NO: 1.

5. The fusion protein according to claim 4, wherein the transport peptide is a C-terminal sequence on the toxic protein portion.

6. The fusion protein according to any one of claims 1-5, wherein the toxic protein is a variant of Pseudomonas Exotoxin A (ETA′).

7. The fusion protein according to claim 6, wherein the ETA′ is lacking a binding domain.

8. The fusion protein according to claim 6, wherein the toxic protein portion ETA′ consists of domains II and III of the Pseudomonas toxin.

9. The fusion protein according to claim 1, wherein the scFv antibody has a binding affinity of 1×10⁻⁶ M, or higher affinity for CD33.

10. The fusion protein according to claim 1, wherein the scFv antibody has a binding affinity of 1×10⁻⁷M or higher affinity for CD33.

11. A pharmaceutical composition, comprising the fusion protein of any one of claims 1-10, and a pharmaceutically-acceptable carrier.

12. A method of treating a disease associated with cells expressing a CD33 cell surface antigen, comprising:
administering to a patient a therapeutically effective amount of a composition of a fusion protein comprising an single chain scFv antibody portion which specifically binds to CD33 and is capable of being internalized into a CD33 expressing cell, and a toxic protein portion.

13. The method according to claim 12, wherein the fusion protein further comprises a transport peptide which facilitates movement into a cell's endoplasmic reticulum.

14. The method according to claim 13, wherein the transport peptide comprises the tetrapeptide KDEL.

15. The method according to any one of claims 12-14, wherein the toxic protein is a variant of Pseudomonas Exotoxin A (ETA′).

16. The method according to claim 15, wherein the ETA′ is lacking a binding domain for □2-macroglobulin receptor.

17. The method according to claim 15, wherein the toxic protein portion ETA′ consists of domains II and III of the Pseudomonas toxin.

18. The method according to claim 12, wherein the scFv has a binding affinity of 1×10⁻⁶ M or higher affinity for CD33.

19. The method according to claim 12, wherein the scFv has a binding affinity of 1×10⁻⁷ M or higher affinity for CD33.

20. The method according to any one of claims 12-19, wherein the disease is acute myeloid leukemia (AML).

21. The method according to claim 20, wherein the patient has relapsing form of AML.

22. The method according to claim 20, wherein the disease is pediatric acute lymphoblastic leukemia (ALL).

23. The method according to any one of claims 20 to 22, further comprising: repeatedly administering the composition to the patient over a period of time and monitoring patient responsiveness over the period of time.

24. A method of inducing apoptosis of human cells, comprising:
contacting a cell line selected from the group consisting of U937, HL-60 and THP-1 with a fusion protein, wherein the fusion protein comprises an antibody fragment portion which binds to any of the cell lines U937, HL-60 and THP-1 with a binding affinity of 1×10⁻⁶ M or higher affinity and is internalized into the cell; and a toxic protein portion linked to the antibody portion, wherein the toxic protein portion further comprises a tetrapeptide KDEL.

25. The method according to claim 24, wherein the antibody fragment portion binds to CD33 with a binding affinity of about 1×10⁻⁷ M or higher affinity.

26. The method according to any one of claims 24 and 25, wherein the toxic protein portion is a variant of a Pseudomonas Exotoxin A (ETA′).

27. The method according to claim 26, wherein the ETA′ lacks the authentic binding domain of the intact ETA.

28. The method according to any one of claims 24-27, wherein the fusion protein is contacted with the cells at a concentration in the range of about 50 ng/ml to about 2,000 ng/ml.

29. A formulation for treating a disease associated with cells expressing a CD33 cell surface antigen, comprising: a pharmaceutically acceptable injectable carrier; and an fusion protein comprising:
an single chain scFv antibody fragment which binds CD33 with a binding affinity of 1×10⁻⁶ M or higher affinity and is capable of being internalized into the CD33 expressing cell;
a portion comprising a variant of Pseudomonas Exotoxin A (ETA′) which lacks a binding domain; and
a C-terminal sequence of SEQ ID NO:1; wherein the fusion protein is present in the formulation in a concentration of about 0.1 mg/ml to about 100 mg/ml.

30. An internalizing fusion protein, comprising: a single chain scFv antibody fragment which binds to CD33 and is capable of being internalized into a CD33 expressing cell, comprising antibody variable domains of a heavy chain and a light chain stabilized by a disulfide bond between the heavy and the light chain; and
a toxic protein portion.

31. The fusion protein according to claim 30, wherein the toxic protein is linked to the antibody fragment.

32. The fusion protein according to claim 30, wherein the toxic protein portion comprises a C-terminal sequence which facilitates movement into a cell's endoplasmic reticulum.

33. The fusion protein according to claim 30, wherein the C-terminal sequence is SEQ ID NO:1, and the toxic protein is a variant of Pseudomonas Exotoxin A (ETA′).

34. The fusion protein according to claim 33, wherein the modified toxic protein portion ETA′ is lacking a binding domain.

35. A internalizing fusion protein comprising an antibody fragment portion which fragment is a single chain scFv antibody fragment which binds to a cell surface antigen CD33 and is characterized by being internalized into a CD33 expressing cell; a modified toxic protein portion bound to the antibody fragment portion by a peptide bond; and a C-terminal sequence which facilitates movement into a cell's endoplasmic reticulum; for use in treating a disease associated with cells expressing the CD33 cell surface antigen.

36. The fusion protein of claim 35, wherein said disease is selected from acute myeloid leukemia (AML) and pediatric acute lymphoblastic leukemia (ALL).

37. Use of an internalizing fusion protein comprising:
an single chain scFv antibody fragment portion which binds to a cell surface antigen CD33 and is capable being internalized into a CD33 expressing cell;
a modified toxic protein portion linked to the antibody fragment portion, and
a C-terminal sequence which facilitates movement into a cell's endoplasmic reticulum;
for the manufacture of a medicament for treating a disease associated with cells expressing a CD33 cell surface antigen.

38. The use of claim 37, wherein said disease is selected from acute myeloid leukemia (AML) and pediatric acute lymphoblastic leukemia (ALL).
